(19) United States
(12) Patent Application Publication
(10) Pub. No.: US 2026/0308690 A1
(43) Pub. Date: Oct. 8, 2026

(54) Epigenetic Signature of T Cell Aging and Malignancy

(71) Applicants: ST. JUDE CHILDREN'S RESEARCH HOSPITAL., INC., Memphis, TN (US); REGENTS OF THE UNIVERSITY OF MINNESOTA, Minneapolis, MN (US)

(72) Inventors: Benjamin A. Youngblood, Memphis, TN (US); Caitlin C. Zebley, Memphis, TN (US); Tian Mi, Memphis, TN (US); David Masopust, New Brighton, MN (US); Andrew G. Soerens, Minneapolis, MN (US)

(21) Appl. No.: 19/482,369

(22) PCT Filed: May 6, 2024
(86) PCT No.: PCT/US2024/027942

§ 371 Date: Nov. 7, 2025

Related U.S. Application Data
(60) Provisional application No. 63/502,532, filed on May 16, 2023.

(51) Int. Cl. C12Q 1/6886

(52) U.S. Cl. CPC C12Q 1/6886; C12Q 2600/112; C12Q 2600/154

(57) ABSTRACT

A 139-gene epigenetic signature for use in. methods for assessing the age and longevity of a T cell, selecting a T cell for a T cell therapy, and/or determining a T-cell acute lymphoblastic leukemia subtype is provided.

### INTRODUCTION

This application claims the benefit from U.S. provisional patent application Ser. No. 63/502,532 filed May 16, 2023, the content of which is incorporated herein by reference in its entirety.

This invention was made with government support under grant nos. AI114442, CA237311, AI144616, AI084913, AI146032, and CA238439 awarded by the National Institutes of Health. The government has certain rights in this invention.

### STATEMENT REGARDING ELECTRONIC FILING OF A SEQUENCE LISTING

A Sequence Listing in XML text format, submitted under 37 C.F.R. § 1.821-1.834, entitled “SJ0109WO_ST26,” 1,970 bytes in size, generated May 3, 2024, and filed via Patent Center, is provided in lieu of a paper copy. This Sequence Listing is hereby incorporated by reference into the specification.

### BACKGROUND

Replicative senescence has been conceptualized as an unavoidable consequence of aging, and thus has been inferred as a metric of cellular lifespan. As such, aging is often described as a path toward proliferative dysfunction, biasing the development of aging metrics toward a count-down to the end of life, rather than counting up and tallying past experiences. As such, accumulation of age-acquired genomic alterations that limit entry into the cell cycle have been reported as a surrogate for cellular age. Specifically, products of the Cdkn2a/2b gene cluster that regulate RB and p53 are elevated in somatic cells of aged mice, linking these genes with age-associated senescence. Further supporting this link, it has been shown that conditional deletion of the gene encoding p16 (INK4a) within this cluster in adult mice significantly prolongs their lifespan. In humans, increased expression of senescence-associated gene products in somatic tissues and peripheral blood similarly correlates with organismal aging. While these cell-cycle regulatory principles can define a finite lifespan of somatic cells, memory T cells have an inherent ability to preserve telomere length during rapid and sequential rounds of antigen-driven replication. The long-lived nature of memory T cells underscores conceptual and logistical challenges of using expression of senescence-associated gene products as an assumptive of T cell age. The extraordinary longevity of immunological memory also raises the question of whether T cell experiential aging (including antigen and cytokine driven signaling events that induce proliferation) can supersede organismal lifespan.

Recent description of a multi-lifetime (ML) murine model of memory T cell differentiation demonstrated that T cells iteratively boosted and adoptively transferred over a 10-year period retained the ability to undergo antigen-driven proliferative bursts through 51 successive rounds of stimulation. In other words, the daughter cells were able to evade replicative senescence without malignant transformation in a setting that far exceeded the organismal lifespan, highlighting the open question of whether cells that experience extraordinary longevity without malignant transformation do so by suspending the aging process. Recent attempts to define molecular clocks to predict organismal age have established epigenetic modifications as an age-estimator with remarkable accuracy. However, these clocks are not tied to any known biological property and have not been assessed in settings of cellular longevity that exceed organismal lifespan. While a relationship between human chronologic age and T cell epigenetics has been described (Goronzy et al. (2018) J. Leukoc. Biol. 104:691-699), which documents an enrichment of memory-associated epigenetic programs in aged populations, there remains a need in the art for an epigenetic signature that takes into consideration the experiential component of T cell aging uncoupled from memory T cell proliferative decline as well as the decline in naïve T cell output due to thymic involution. The present invention meets this need in the art.

### SUMMARY OF THE INVENTION

This invention provides methods for assessing the age and longevity of a T cell, selecting a T cell for a T cell therapy, and/or determining a T-cell acute lymphoblastic leukemia (T-ALL) subtype by determining an average methylation of a plurality of genes in the T cell, the plurality of genes selected from the group consisting of: Ebf3, Irx5, Pou3f3, Irs2, Irx3, Ctbp2, Efna5, Tle1, Sall1, Foxd3, Zfp423, Hmga2, Sox1, Afdn, Nr2f2, Nkx6-1, Zfp316, Kctd15, Sox11, Sox4, Spry2, Cbln1, Tshz3, Zfp503, Tfap2c, Inhbb, Irx2, Gas1, Fat4, Nkx2-4, Zcchc14, Cadm1, Pcdh10, Onecut2, Prdm6, Mycn, Fzd8, Epha7, Sox9, Nkx2-1, Epb4114b, Dio3, Otx1, Zbtb10, Jag1, Nkx6-2, Tbx3, Tbx18, Agap1, Foxc2, Sox2, Foxe1, Fzd1, Insm2, Adra2c, Gm13375, Nfib, Foxg1, Ptpn3, Mtcl1, Ube2ql1, Zfp536, AI606473, Pitx2, Zfp521, Grb10, Lin28b, Gpr27, Cacna2d3, Zic4, Fam78b, Begain, Gm9748, Gm5468, Prox1, Schip1, Bhlhe22, Cdh2, Fat1, Twist1, Ptprg, Sdk2, Bmp4, Mn1, Gm30731, Nova1, Grid1, Zfhx3, Gli3, Magi1, Pax3, Ccdc85c, Bnc2, Mkx, Id4, Auts2, Tlx3, Shisa6, Ctnnd2, Trank1, Fam19a5, Chsy3, Lrp12, Hoxd8, Lrig1, Tenm4, Flt1, Kcnn2, Pcdh17, Reln, Pou3f1, Lrrc4, Chst15, Large1, Fezf1, Gab1, Plcb4, Cdh4, Cilp2, Prickle1, Sox3, Smco4, Fgfr2, Hoxd11, Skor2, Fignl2, Cnr1, Nbea, Trim71, Snap91, Sox14, Kcnk9, Mpped2, Sema6a, Pde5a, Wnt5a, Grip1, Dmrt3, and Adgrl3, wherein an average hypermethylation of the plurality of genes in the T cell as compared to a control naïve T cell is indicative of the age and longevity of the T cell, use of the T cell in a T cell therapy, and/or the T-ALL subtype. In one aspect, the T cell is a memory T cell. In another aspect, the methylation of the plurality of genes is determined in the gene body of the plurality of genes. In a further aspect, the methylation of the plurality of genes is determined in CpG islands and shores of the plurality of genes. In aspects pertaining to the selection of a T cell therapy, the T cell may have at least one edited gene. In aspects pertaining to determining a T-ALL subtype (e.g., a HOXA or TLX3 subtype), the method may further include the step of determining a methylation status of a Cdkn2a or Cdkn2b promoter.

### BRIEF DESCRIPTION OF THE DRAWINGS

FIG. 1 illustrates the schema for generating multi-lifetime memory CD8 T cells. Naïve mice were infected with Vesicular Stomatitis Virus (VSV) generating antigen-specific memory CD8 T cells. Heterologous VSV infection and boosting of the memory T cells was performed after 30 versus 60 days post previous infection. After three infections, antigen-specific memory CD8 T cells were transferred into congenically distinct naïve mice, and the boosting strategy was repeated over a time frame that yielded memory T cells with age ranging between 0.5 to ~4 mouse lifetime (LT) equivalents. Young memory n=6, 0.5×LT n=3, 2-3×LT n=5, 4×LT n=3.

FIG. 2 shows a principal component analysis (PCA) plot of the 3000 most variable CpG sites comparing multi-lifetime and ‘young’ memory T cells.

FIG. 3 is a summary graph showing mean methylation across the top multi-lifetime gain of methylation of differentially methylated regions (DMRs) relative to boosting and age of memory T cells.

FIG. 4 is a summary graph of total DMR methylation among the Cdkn2a/b loci for young and multi-lifetime memory CD8 T cells.

FIG. 5 is a summary graph of average multi-lifetime-‘experiential age’ (ML-EA)-associated methylation levels for human naïve, central memory (Tcm), effector memory (Tem), and CMV-specific memory T cell samples.

FIG. 6 are summary box plots (FIGS. 6A, 6B, 6C) for T-ALL patient tumor genome-wide DNA methylation levels versus EA programs among three independent cohorts (GRAALL n=143 (6A), St. Jude/ECOG n=48 (6C), UTokyo n=98 (6B)).

### DETAILED DESCRIPTION OF THE INVENTION

The present invention provides an epigenetic signature composed of 139 genes for use in evaluating the age of T cells. This epigenetic signature is enriched for genes controlling cell-cycle and predicts an accelerated age estimate for several T cell acute lymphoblastic leukemia subtypes (T-ALL). Moreover, T-ALL subtypes can be delineated with the epigenetic signature. Additionally, the epigenetic signature is specific for T cells, is associated with known cell cycle regulatory loci, is correlated with proliferative experience through at least four successive lifetimes and is distinct from malignancy associated epigenetic programs allowing for the delineation of long-lived T cells from malignant T cells. Accordingly, in addition to being useful in therapeutic settings that are attempting to reverse the aging process or in the development of senolytic therapies, the epigenic signature described herein may be used in assessing the longevity versus malignancy potential of T cells that have been gene edited for therapy thereby ensuring that the T cells have not acquired malignancy-associated programs. Advantageously, the epigenetic signature allows for better interpretation of the estimated age of T cells when applied to therapeutic settings that modulate cell cycle entry.

Thus, the present invention relates to methods for assessing the age and longevity of a T cell, selecting a T cell for a T cell therapy and determining a T-cell acute lymphoblastic leukemia (T-ALL) subtype by determining or measuring the average DNA methylation of the 139-gene epigenetic signature described herein. In accordance with the methods of the invention, a T cell or population of T cells is obtained, the DNA methylation status of the 139 genes of the 139-gene epigenetic signature is measured or determined and the average DNA methylation of the 139-gene epigenetic signature as compared to the average DNA methylation of a control naïve T cell is indicative of the age of the T cell and therefore its longevity, use in a T cell therapy, and particular subtype of T-ALL.

In accordance with the methods of this invention, the plurality of genes of the epigenetic signature and chromosomal location of the same in murine DNA are provided in Table 1.

TABLE 1

| Murine | | | | Human | | |
|---|---|---|---|---|---|---|
| Chr | Start | End | Gene | Chr | Start | End |
| chr7 | 137312322 | 137323391 | Ebf3 | chr10 | 131760410 | 131771883 |
| chr8 | 92354960 | 92367384 | Irx5 | chr16 | 54961714 | 54974421 |
| chr1 | 42693264 | 42700906 | Pou3f3 | chr2 | 105468053 | 105475675 |
| chr8 | 11006915 | 11010370 | Irs2 | chr13 | 110436875 | 110440703 |
| chr8 | 91799220 | 91807364 | Irx3 | chr16 | 54317971 | 54326318 |
| chr7 | 133121437 | 133125028 | Ctbp2 | chr10 | 126847122 | 126850834 |
| chr17 | 62879456 | 62884695 | Efna5 | chr5 | 107004657 | 107009959 |
| chr4 | 72198943 | 72202226 | Tle1 | chr9 | 84301900 | 84305951 |
| chr8 | 89040711 | 89047578 | Sall1 | chr16 | 51183327 | 51190495 |
| chr4 | 99652364 | 99658558 | Foxd3 | chr1 | 63784388 | 63790682 |
| chr8 | 87957731 | 87962812 | Zfp423 | chr16 | 49888009 | 49893347 |
| chr10 | 120474001 | 120478566 | Hmga2 | chr12 | 66216608 | 66221095 |
| chr8 | 12394768 | 12398436 | Sox1 | chr13 | 112720378 | 112724067 |
| chr17 | 13759677 | 13762152 | Afdn | chr6 | 168225861 | 168229303 |
| chr7 | 70357802 | 70363170 | Nr2f2 | chr15 | 96872485 | 96877793 |
| chr5 | 101662498 | 101669890 | Nkx6-1 | chr4 | 85417502 | 85425388 |
| chr5 | 143252682 | 143255885 | 2fp316 | chr7 | 6691680 | 6694930 |
| chr7 | 34652615 | 34656021 | Kctd15 | chr19 | 34286088 | 34289640 |
| chr12 | 27338445 | 27343890 | Sox11 | chr2 | 5830992 | 5838300 |
| chr13 | 28949243 | 28954370 | Sox4 | chr6 | 21593327 | 21598596 |
| chr14 | 105894607 | 105899428 | Spry2 | chr13 | 80912687 | 80917507 |
| chr8 | 87468415 | 87473928 | Cbln1 | chr16 | 49311309 | 49317635 |
| chr7 | 36693725 | 36698983 | Tshz3 | chr19 | 31839355 | 31844753 |
| chr14 | 21984813 | 21990067 | Zfp503 | chr10 | 77158428 | 77163793 |
| chr2 | 172546635 | 172552413 | Tfap2c | chr20 | 55201281 | 55207227 |
| chr1 | 119419435 | 119423115 | Inhbb | chr2 | 121101655 | 121105304 |
| chr13 | 72627124 | 72632154 | Irx2 | chr5 | 2748273 | 2753633 |
| chr13 | 60175205 | 60178800 | Gas1 | chr9 | 89560029 | 89563686 |
| chr3 | 38884499 | 38888621 | Fat4 | chr4 | 126234790 | 126239228 |
| chr2 | 147083384 | 147086243 | Nkx2-4 | chr20 | 21376058 | 21379000 |
| chr8 | 121651372 | 121653961 | Zcchc14 | chr16 | 87524689 | 87527730 |
| chr9 | 47529197 | 47533100 | Cadm1 | chr11 | 115372419 | 115376305 |
| chr3 | 45375711 | 45382533 | Pcdh10 | chr4 | 134067774 | 134074529 |
| chx18 | 64338842 | 64346797 | Onecut2 | chr18 | 55101481 | 55109153 |
| chr18 | 53460589 | 53465998 | Prdm6 | chr5 | 122421811 | 122427231 |
| chr12 | 12936889 | 12943200 | Mycn | chr2 | 16079190 | 16086333 |
| chr18 | 9211300 | 9215038 | Fzd8 | chr10 | 35928213 | 35932171 |
| chr4 | 28812487 | 28815815 | Epha7 | chr6 | 94126176 | 94129928 |
| chr11 | 112779703 | 112785578 | Sox9 | chr17 | 70114640 | 70120569 |
| chr12 | 56532730 | 56538017 | Nkx2-1 | chr14 | 36986306 | 36991477 |
| chr4 | 57141261 | 57144023 | Epb41l4b | chr9 | 112080713 | 112083646 |
| chr12 | 110277178 | 110280728 | Dio3 | chr14 | 102025684 | 102029379 |
| chr11 | 21992772 | 21998795 | Otx1 | chr2 | 63280847 | 63287543 |
| chr3 | 9249679 | 9252235 | Zbtb10 | chr8 | 81397508 | 81400168 |
| chr2 | 137114069 | 137117851 | Jag1 | chr20 | 10651955 | 10655839 |
| chr7 | 139580680 | 139583411 | Nkx6-2 | chr10 | 134597910 | 134600018 |
| chr5 | 119666987 | 119674447 | Tbx3 | chr12 | 115118533 | 115126214 |
| chr9 | 87729089 | 87734215 | Tbx18 | chr6 | 85471834 | 85477651 |
| chr1 | 89454027 | 89456122 | Agap1 | chr2 | 236401793 | 236404051 |
| chr8 | 121114623 | 121118482 | Foxc2 | chr16 | 86598823 | 86602905 |
| chr3 | 34646990 | 34652291 | Sox2 | chr3 | 181426600 | 181432052 |
| chr4 | 46342923 | 46345892 | Foxe1 | chr9 | 100614630 | 100617916 |
| chr5 | 4755896 | 4758869 | Fzd1 | chr7 | 90892855 | 90895895 |
| chr12 | 55598644 | 55601000 | Insm2 | chr14 | 36002431 | 36005210 |
| chr5 | 35278443 | 35281232 | Adra2c | chr4 | 3766859 | 3769692 |
| chr2 | 20967664 | 20970048 | Gm13375 | chr10 | 25011126 | 25014126 |
| chr4 | 82503966 | 82507789 | Nfib | chr9 | 14312668 | 14316890 |
| chr12 | 49381657 | 49386279 | Foxg1 | chr14 | 29233399 | 29238304 |
| chr4 | 57299485 | 57302203 | Ptpn3 | chr9 | 112259637 | 112262402 |
| chr17 | 66448061 | 66450736 | Mtcl1 | chr18 | 8704169 | 8707013 |
| chr13 | 69738161 | 69741083 | Ube2ql1 | chr5 | 6447033 | 6449852 |
| chr7 | 37768636 | 37773913 | Zfp536 | chr19 | 30715342 | 30720512 |
| chr3 | 154327250 | 154334232 | AI606473 | chr1 | 75596228 | 75603769 |
| chr3 | 129212631 | 129219354 | Pitx2 | chr4 | 111538821 | 111545493 |
| chr18 | 13969913 | 13973528 | Zfp521 | chr18 | 22929475 | 22932971 |
| chr11 | 12035916 | 12038364 | Grb10 | chr7 | 50859640 | 50862202 |
| chr10 | 45485271 | 45487213 | Lin28b | chr6 | 105387676 | 105389988 |
| chr6 | 99691832 | 99693793 | Gpr27 | chr3 | 71802338 | 71804303 |
| chr14 | 29720258 | 29723429 | Cacna2d3 | chr3 | 54154946 | 54158232 |
| chr9 | 91377392 | 91384522 | Zic4 | chr3 | 147108548 | 147115558 |
| chr1 | 167000291 | 167002825 | Fam78b | chr1 | 166134197 | 166136780 |
| chr12 | 109052224 | 109054077 | Begain | chr14 | 101033434 | 101035672 |
| chr14 | 79767810 | 79772117 | Gm9748 | chr13 | 53419150 | 53423611 |
| chr15 | 25411887 | 25416183 | Gm5468 | chr5 | 17215138 | 17219656 |
| chr1 | 190168527 | 190175684 | Prox1 | chr1 | 214155970 | 214163564 |
| chr3 | 68493241 | 68496014 | Schip1 | chr3 | 159480815 | 159483706 |
| chr3 | 18053384 | 18056209 | Bhlhe22 | chr8 | 65491910 | 65494847 |
| chr18 | 16806811 | 16810047 | Cdh2 | chr18 | 25755370 | 25758707 |
| chr8 | 44934164 | 44939008 | Fat1 | chr4 | 187644084 | 187648946 |
| chr12 | 33955456 | 33959105 | Twist1 | chr7 | 19155824 | 19159466 |
| chr14 | 11551647 | 11556952 | Ptprg | chr3 | 61544814 | 61550873 |
| chr11 | 114064634 | 114067662 | Sdk2 | chr17 | 71638977 | 71642029 |
| chr14 | 46385572 | 46390993 | Bmp4 | chr14 | 54418468 | 54423945 |
| chr5 | 111416456 | 111420699 | Mn1 | chr22 | 28193943 | 28198640 |
| chr4 | 22486862 | 22492359 | Gm30731 | chr6 | 99278799 | 99284013 |
| chr12 | 46817439 | 46820202 | Nova1 | chr14 | 27065194 | 27068516 |
| chr14 | 34818775 | 34821679 | Grid1 | chr10 | 88124629 | 88127640 |
| chr8 | 108703648 | 108705440 | Zfhx3 | chr16 | 73091273 | 73093025 |
| chr13 | 15462497 | 15465388 | Gli3 | chr7 | 42275130 | 42278490 |
| chr6 | 94282204 | 94285427 | Magi1 | chr3 | 66022827 | 66026475 |
| chr1 | 78215628 | 78218363 | Pax3 | chr2 | 223182674 | 223185528 |
| chr12 | 108273967 | 108276617 | Ccdc85c | chr14 | 100069057 | 100071846 |
| chr4 | 84674003 | 84676435 | Bnc2 | chr9 | 16869551 | 16872340 |
| chr18 | 7000992 | 7006267 | Mkx | chr10 | 28030605 | 28036473 |
| chr13 | 48260349 | 48263317 | Id4 | chr6 | 19836807 | 19839758 |
| chr5 | 132540210 | 132544850 | Auts2 | chr7 | 69062003 | 69066155 |
| chr11 | 33199742 | 33204551 | Tlx3 | chr5 | 170735265 | 170740246 |
| chr11 | 66524948 | 66527282 | Shisa6 | chr17 | 11143248 | 11145755 |
| chr15 | 30172190 | 30174122 | Ctnnd2 | chr5 | 11902968 | 11905002 |
| chr9 | 111311258 | 111312989 | Trank1 | chr3 | 36985299 | 36987040 |
| chr15 | 87623578 | 87625985 | Fam19a5 | chr22 | 48970434 | 48972892 |
| chr18 | 59174618 | 59176865 | Chsy3 | chr5 | 129239386 | 129241717 |
| chr15 | 39942643 | 39944695 | Lrp12 | chr8 | 105600043 | 105602266 |
| chr2 | 74704470 | 74706194 | Hoxd8 | chr2 | 176994013 | 176995728 |
| chr6 | 94699047 | 94701469 | Lrig1 | chr3 | 66550252 | 66553110 |
| chr7 | 96209159 | 96214038 | Tenm4 | chr11 | 79147982 | 79152896 |
| chr5 | 147724095 | 147727750 | Flt1 | chr13 | 29067176 | 29071064 |
| chr18 | 45558097 | 45561300 | Kenn2 | chr5 | 113696412 | 113699682 |
| chr14 | 84444145 | 84448835 | Pcdh17 | chr13 | 58204828 | 58209423 |
| chr5 | 22343668 | 22345737 | Reln | chr7 | 103629009 | 103631287 |
| chr4 | 124656385 | 124660195 | Pou3f1 | chr1 | 38509953 | 38513743 |
| chr6 | 28831851 | 28833840 | Lrrc4 | chr7 | 127670928 | 127672851 |
| chr7 | 132315285 | 132317710 | Chst15 | chr10 | 125851101 | 125854021 |
| chr8 | 73352315 | 73355217 | Large1 | chr22 | 34315832 | 34318856 |
| chr6 | 23244641 | 23251219 | Fezf1 | chr7 | 121940950 | 121947867 |
| chr8 | 80878579 | 80881102 | Gab1 | chr4 | 144256753 | 144259411 |
| chr2 | 135658239 | 135660980 | Plcb4 | chr20 | 9048168 | 9051164 |
| chr2 | 179441402 | 179443916 | Cdh4 | chr20 | 59826208 | 59829224 |
| chr8 | 69880808 | 69883344 | Cilp2 | chr19 | 19654169 | 19656876 |
| chr15 | 93594658 | 93596736 | Prickle1 | chr12 | 42982075 | 42984552 |
| chrX | 60890671 | 60894611 | Sox3 | chrX | 139584454 | 139588421 |
| chr9 | 15493036 | 15494541 | Smco4 | chr11 | 93275181 | 93276683 |
| chr7 | 130263828 | 130266872 | Fgfr2 | chr10 | 123355463 | 123358640 |
| chr2 | 74681447 | 74684021 | Hoxd11 | chr2 | 176971094 | 176973753 |
| chr18 | 76858218 | 76861171 | Skor2 | chr18 | 44772952 | 44775887 |
| chr15 | 101052605 | 101054497 | Fignl2 | chr12 | 52214375 | 52216349 |
| chr4 | 33923442 | 33925465 | Cnr1 | chr6 | 88875218 | 88877469 |
| chr3 | 56182345 | 56184363 | Nbea | chr13 | 35515531 | 35517965 |
| chr9 | 114562085 | 114564516 | Trim71 | chr3 | 32857927 | 32860452 |
| chr9 | 86879221 | 86881408 | Snap91 | chr6 | 84417326 | 84419844 |
| chr9 | 99868100 | 99873568 | Sox14 | chr3 | 137485806 | 137491487 |
| chr15 | 72545661 | 72548698 | Kcnk9 | chr8 | 140714592 | 140717300 |
| chr2 | 106692996 | 106696085 | Mpped2 | chr11 | 30605270 | 30608390 |
| chr18 | 47366219 | 47371708 | Sema6a | chr5 | 115907947 | 115913817 |
| chr3 | 122728746 | 122731083 | Pde5a | chr4 | 120547844 | 120550356 |
| chr14 | 28504098 | 28507916 | Wnt5a | chr3 | 55519044 | 55523029 |
| chr10 | 119452745 | 119455898 | Grip1 | chr12 | 67461331 | 67464545 |
| chr19 | 25608690 | 25611934 | Dmrt3 | chr9 | 974818 | 978191 |
| chr5 | 81019370 | 81023012 | Adgrl3 | chr4 | 62065675 | 62069211 |

The term “DNA methylation” refers to the covalent attachment of a methyl group to DNA at the C5 position of a cytosine ring. In mammals, DNA methylation typically occurs at a cytosine (C) that is followed, in the 5′ to 3′ direction, by a guanine (G). This dinucleotide is often referred to as a CpG. The term “level of methylation,” “degree of methylation,” “level of DNA methylation,” “degree of DNA methylation” or “methylation status” refers to the proportion of cytosine nucleotide residues that are methylated within a given region of DNA, i.e., the total number of methylated cytosine residues in the region by the total number of nucleotides in the region. DNA methylation in mammals occurs most frequently on cytosines in CpG dinucleotides, and the level of methylation is often the same or about the same as the level of CpG methylation. Where the present disclosure refers to a level of methylation, certain embodiments relate specifically to the level of CpG methylation, i.e., the number of CpGs in the region that are methylated on the cytosine residue divided by the total number of CpGs in the region. DNA methylation levels may be expressed, e.g., in terms of beta-values of between 0 and 1, which may be interpreted as the approximation of the percentage of methylation for the population of a given CpG site in a sample. Alternatively, M-values may be used, wherein a positive M-value means that more DNA molecules are methylated than unmethylated, while negative M-values mean the opposite.

The term “hypermethylation” refers to a higher average level of methylation than the average level of methylation of a control level or an increased level of methylation as compared with a particular level with which it is compared. For example, if a particular region or plurality of regions of genomic DNA has a level of methylation of 70% in memory T cells and normally has a level of methylation of 10% in naïve T cells, the DNA region is considered to be hypermethylated in the memory T cells as compared to the naïve T cells.

In some aspects, a T cell used in the methods of this invention is provided in a population of cells, e.g., peripheral blood mononuclear cells, or population of isolated T cells, e.g., two or more T cells isolated by cell sorting. In another aspect, the T cell is an individual T cell, e.g., a T cell isolated by cell sorting. The term “isolated” means 1) separated from at least some of the components with which it is usually associated in nature; 2) prepared or purified by a process that involves the hand of man; and/or 3) not occurring in nature, e.g., present in an artificial environment. In some aspects, an isolated cell is a cell that has been removed from a subject, generated in vitro, separated from at least some other cells in a cell population or sample, or that remains after at least some other cells in a cell population or sample have been removed or eliminated.

A T cell or population of T cells of use in the methods of this invention may be identified by cell surface markers conventionally used in the art. In some aspects, the T cell is a memory T cell. In other aspects, the memory T cell is a CD4⁺ memory T cell or CD8⁺ memory T cell. In further aspects, the T cell is a central memory T cell (Tcm), effector memory T cell (Tem) or stem cell-like memory T cell (Tscm). By way of illustration, Tcm cells may be identified as being CCR7⁺ CD45RA⁻ CD45RO⁺ CD62L⁺ CD27⁺. Tem cells may be identified as being CCR7⁻ CD45RA⁻ CD45RO⁺ CD62L⁻ CD27⁻. Tscm may be identified as being CCR7⁺ CD95⁺ CD45RO⁻ CD27⁺ CD45RA⁺ CD62L⁺ CD122⁺. By comparison, naïve T cells (Tn) may be identified as being CD45RA⁺ CCR7⁺ CD45RO⁻ CD27⁺. In some aspects, T cells of use in this invention include the following:

$$CD {8}^{+} CCR {7}^{+} CD 45 {RA}^{-} CD 45 {RO}^{+} CD 62 {L}^{+} CD {27}^{+} T cells , \qquad ( a )$$

$$CD {4}^{+} CCR {7}^{+} CD 45 {RA}^{-} CD 45 {RO}^{+} CD 62 {L}^{+} CD {27}^{+} T cells , \qquad ( b )$$

$$CD {8}^{+} CCR {7}^{-} CD 45 {RA}^{-} CD 45 {RO}^{+} CD 62 {L}^{-} CD {27}^{-} T cells , \qquad ( c )$$

$$CD {4}^{+} CCR {7}^{-} CD 45 {RA}^{-} CD 45 {RO}^{+} CD 62 {L}^{-} CD {27}^{-} T cells , \qquad ( d )$$

$$CD {8}^{+} CCR {7}^{+} CD {95}^{+} CD 45 {RO}^{-} CD {27}^{+} CD 45 {RA}^{+} CD 62 {L}^{+} CD {122}^{+} T cells , and / or \qquad ( e )$$

$$CD {4}^{+} CCR {7}^{+} CD {95}^{+} CD 45 {RO}^{-} CD {27}^{+} CD 45 {RA}^{+} CD 62 {L}^{+} CD {122}^{+} T cells . \qquad ( f )$$

In accordance with the methods of this invention, the average of the degree or level of methylation of the plurality of genes in Table 1 is significantly correlated with T cell age and longevity. In some aspects, the level of DNA methylation of the genes of the 139-gene signature is determined or measured over the entire locus, i.e., the translated and/or untranslated 5′ regulatory regions of the gene. In other aspects, the level of methylation of the plurality of genes is determined or measured for the gene body. As used herein, the term “gene body” refers to the portion of a gene that is transcribed, from the transcription start site to the end of the transcribed region. The term “transcription start site” refers to the DNA nucleotide at which transcription of a RNA begins, i.e., the nucleotide that is transcribed to yield the first ribonucleotide in an RNA transcript. Transcription start sites may be defined based on RefSeq gene annotations.

In some aspects, the methylation of the plurality of genes is determined or measured at CpG islands and shores of the plurality of genes. The term “CpG island” (CGI) refers to a region of genomic DNA that has an elevated G+C content (proportion of nucleotides that are either G or C) as compared with the mammalian genome as a whole, in which CpG dinucleotides are underrepresented. In vertebrates, CpG islands are enriched in certain regions of the genome involved in initiation of gene transcription. CGIs frequently exist in an unmethylated state that is transcriptionally permissive and marked by histone modifications that are characteristic of transcriptionally active chromatin such as histone acetylation (H3/H4Ac) and H3K4me3. While often unmethylated in normal cells, CGIs can become methylated under certain conditions and in certain tissues. DNA methylation of CGIs in promoters is associated with stable long-term silencing.

CGIs may be identified as regions of genomic DNA at least 200 bp in length that have a G+C content of at least 50% and a CpG frequency (observed/expected) of at least 0.6 (Gardiner-Garden & Frommer (1987) J. Mol. Biol. 196:261-282). In some embodiments a CGI is at least 300 bp, at least 400 bp, or at least 500 bp long, e.g., between about 500 bp and about 1 kb, or between about 1 kb and about 2 kb. “CpG island shores” are the regions extending 2 kb on either side of a CpG island. These regions have a lower CpG density than CGIs and harbor numerous tissue-specific differentially methylated regions. A “low CpG region” is a region that has a lower CpG density than that found in CpG islands.

As indicated herein, the plurality of genes provided in Table 1 were shown to be differentially methylated regions of DNA that distinguished young or naïve T cells from T cells that had an advanced experiential age (e.g., T cells with an age of at least 0.5, 1, 2, 3, 4, 10, 50, 100 or more lifetime equivalents). For the purposes of this invention, the term “differentially methylated region” (DMR) refers to a region of genomic DNA that is differentially marked or has a differential level of DNA methylation in T cells having an advanced experiential age as compared to young or naïve T cells. In some aspects, a difference is about 5%, 10%, 15%, 20%, 25%, 30%, 40%, 50%, 60%, 70%, 80%, 90% in the level of methylation of a T cell having an advanced experiential age as compared to a young or naïve T cell. In other aspects, a change is an increase by about 5%, 10%, 15%, 20%, 25%, 30%, 40%, 50%, 60%, 70%, 80%, 90% from a level of about 5%, 10%, 15%, 20%, 25%, 30%, 40%, 50%, 60%, 70%, 80%, 90% methylation (up to a maximum of 100% methylation).

The level of methylation of the plurality of genes provided in Table 1 may be determined by any suitable technique conventionally used in the art for assessing, determining or measuring DNA methylation. For example, commercial array platforms (see, e.g., US 2006/0292585) or sequencing approaches of individual genes may be used. This includes standard lab techniques or array platforms. A variety of methods for detecting methylation status or patterns have been described in, for example U.S. Pat. Nos. 6,214,556, 5,786,146, 6,017,704, 6,265,171, 6,200,756, 6,251,594, 5,912,147, 6,331,393, 6,605,432, 6,300,071, US 2003/0148327, US 2003/0148326, US 2003/0143606, US 2003/0082609 and US 2005/0009059. For a review of some methylation detection methods, see Oakeley (1999) Pharmacology & Therapeutics 84:389-400. Available methods include, but are not limited to: reverse-phase HPLC, thin-layer chromatography, SssI methyltransferases with incorporation of labeled methyl groups, the chloracetaldehyde reaction, differentially sensitive restriction enzymes, hydrazine or permanganate treatment (m5C is cleaved by permanganate treatment but not by hydrazine treatment), sodium bisulfite, combined bisulphate-restriction analysis, and methylation sensitive single nucleotide primer extension.

To quantify the methylation level, one may follow the standard protocol described by ILLUMINA™ to calculate the beta value of methylation, which equals the fraction of methylated cytosines in that location. Other suitable methods for quantifying DNA methylation include, for example:
a) Molecular break light assay for DNA adenine methyltransferase activity is an assay that is based on the specificity of the restriction enzyme DpnI for fully methylated (adenine methylation) GATC sites in an oligonucleotide labeled with a fluorophore and quencher. The adenine methyltransferase methylates the oligonucleotide making it a substrate for DpnI. Cutting of the oligonucleotide by DpnI gives rise to a fluorescence increase.
b) Methylation-Specific Polymerase Chain Reaction (PCR) is based on a chemical reaction of sodium bisulfite with DNA that converts unmethylated cytosines of CpG dinucleotides to uracil or UpG, followed by traditional PCR. However, methylated cytosines will not be converted in this process, and thus primers are designed to overlap the CpG site of interest, which allows one to determine methylation status as methylated or unmethylated. The beta value can be calculated as the proportion of methylation.
c) Whole genome bisulfite sequencing, also known as BS-Seq, is a genome-wide analysis of DNA methylation. It is based on the sodium bisulfite conversion of genomic DNA, which is then sequencing on a Next-Generation Sequencing (NGS) platform. The sequences obtained are then re-aligned to the reference genome to determine methylation states of CpG dinucleotides based on mismatches resulting from the conversion of unmethylated cytosines into uracil.
d) The Hpall tiny fragment Enrichment by Ligation-mediated PCR (HELP) assay is based on restriction enzymes' differential ability to recognize and cleave methylated and unmethylated CpG DNA sites.
e) Methyl Sensitive Southern Blotting is similar to the HELP assay but uses Southern blotting techniques to probe gene-specific differences in methylation using restriction digests. This technique is used to evaluate local methylation near the binding site for the probe.
f) Pyrosequencing of bisulfite treated DNA is a sequencing of an amplicon made by a normal forward primer but a biotinylated reverse primer to PCR the gene of choice. The Pyrosequencer then analyses the sample by denaturing the DNA and adding one nucleotide at a time to the mix according to a sequence given by the user. If there is a mismatch, it is recorded and the percentage of DNA for which the mismatch is present is noted. This gives the user a percentage methylation per CpG island.

Any statistical approach can be used to relate the methylation levels to T cell age and longevity. Using conventional regression model/analysis tools and methodologies known in the art, a number of age prediction models are contemplated for use with specific genomic DNA samples and/or specific analysis techniques and/or specific individual populations (see, e.g., statistical package R as discussed in Wu et al. (2015) Nucl. Acids Res. 43:e141, doi:10.1093/nar/gkv715).

Estimating T cell age and longevity can be used for both diagnostic and prognostic purposes as well as therapeutic purposes and in the study of the effect of medication, food compounds and/or special diets. In addition, T cell age estimations can be used to assess longevity versus malignancy potential in T cells that have been gene edited for therapy. Further, a measurement of relevant methylation patterns in genomic DNA from T cells also provides a tool in routine medical screening to tailor interventions based on the epigenetic biological age instead of the chronological age. In a further aspect, the methods disclosed herein can be applied to assess the efficacy of a treatment or compound used in reversing aging, rejuvenation, or development of a senolytic therapy.

Accordingly, in one aspect, the present invention provides a method for selecting a T cell for a T cell therapy by determining the average methylation of the 139 gene listed in Table 1 in the T cell, wherein an average hypermethylation of the plurality of genes in the T cell as compared to a control naïve T cell is indicative of the use of the T cell in a T cell therapy. In some aspects, the T cell therapy is an immunotherapy. In other aspects, the T cell therapy is a chimeric antigen receptor (CAR) T cell therapy. In further aspects, the T cell has at least one edited gene. T cells may be edited with conventional CRISPR-Cas9 systems or novel editors that can install desired genetic changes with or without introduction of a double-stranded break (DSB) into the genome. These tools and strategies can be directly applied to targeting negative regulators of T cell function, directing therapeutic transgenes to specific genomic loci, and generating reproducibly safe and potent allogeneic universal CAR T-cell products for on-demand cancer immunotherapy. Advantageously, the epigenetic signature of this invention can delineate long-lived T cells from malignant T cells. Such a method may serve as a ‘safety release’ assay thereby ensuring that the T cells to be used in an immunotherapy have not acquired malignancy-associated programs.

In another aspect, the present invention provides a method for determining a T-ALL subtype by determining the average methylation of the 139 gene listed in Table 1 in the T cell, wherein the average methylation of the plurality of genes in the T cell as compared to a control naïve T cell is indicative of the T-ALL subtype. As demonstrated herein, HOXA and TLX3 subtypes of T-ALL were derived from transformation events that occurred earlier in T-cell development and exhibited T cell age estimates ranging from ~100-200 years old compared to TAL1 subtypes. Accordingly, in some aspects, this method of the invention may be used in differentiating T-ALL subtypes with HOXA and TLX3 genetic mutations from those with TAL1 mutations. In addition, the results presented herein demonstrated that while leukemia and iteratively stimulated human memory T cells have methylated the gene body and exons of the Cdkn2a or Cdkn2b locus, only the leukemia acquired methylation at the promoter region. Thus, in some aspects of this invention, the method further includes the step of determining a methylation status of a Cdkn2a and/or Cdkn2b promoter.

The following non-limiting examples are provided to further illustrate the present invention.

### Example 1: Materials and Methods

Mice and Infection. Donor female B6.SJL-Ptprc^{a}Pepc^{b}/BoyJ (CD45.1+ B6) were bred at the University of Minnesota animal facilities. Female C57BL/6J (CD45.2+ B6) mice were purchased from Jackson Laboratories and served as recipient mice. Animals were treated according to the Institutional Animal Care and Use Committee guidelines. CD45.1+ tertiary memory cells were expanded through heterologous prime-boost-boost infection with 10⁶ plaque forming units (PFU) Vesicular Stomatitis Virus (VSV) subtype New Jersey (NJ), an experiment-specific period of rest, 2×10⁶ PFU Vaccinia Virus (VV) expressing the VSV-Indiana nucleoprotein (N), an experiment-specific period of rest, and 10⁷ PFU of VSV-Indiana (Ind). Upon transfer to recipient CD45.2+ mice, cells were expanded through heterologous prime-boost-boost infection with 10⁶ PFU of VSV-Ind, an experiment-specific period of rest, 2×10⁶ PFU VV-N, an experiment-specific period of rest, and 10⁷ PFU VSV-NJ. All heterologous prime boost-boost infections were delivered via the tail vein.

Mouse T Cell Isolation and Adoptive Transfer. A single cell suspension was prepared from the spleen and macroscopic lymph nodes of donor mice. CD8+ T cells were enriched via negative selection before they were stained with surface antibodies. CD8+ T cell enrichment was done using the EASYSEP™ Mouse CD8+ T Cell Isolation Kit (StemCell Technologies, Cambridge, MA) following manufacturer's instructions or using a prepared cocktail of biotinylated antibodies. Purified CD8+ T Cells were then stained with anti-mouse CD8a (53-6.7) from BD Biosciences, anti-mouse CD45.1 (A20), anti-mouse CD45.2 (104), and GHOST DYE™ viability dye from Tonbo Biosciences, and N-Tetramer. CD8a+, VSV-N-Tetramer+, CD45.1+, CD45.2− cells were sorted on a BD FACS Aria II and 10⁵ sorted cells were transferred via the tail vein into recipient mice. Infections resumed the following day.

Human T Cells. CMV-Specific CD8 T cells were purified via fluorescence activated cell sorting (FACS) from donor blood samples obtained by leukapheresis using an IRB-approved protocol of the Einstein-Rockefeller-CUNY Center for AIDS Research (ERC-CFAR). Peripheral blood mononuclear cells (PBMCs) were isolated from deidentified blood samples using FICOLL® density gradient centrifugation, washed, resuspended in in freezing media (10% dimethyl sulfoxide (DMSO) in fetal bovine serum (FBS)), aliquoted (5×10⁷ cells/mL) and stored at −150° C. Freshly thawed PBMCs from HIV seropositive or HIV seronegative donors were washed with FACS buffer (phosphate-buffered saline (PBS) containing 5% FBS and 1 mg/mL sodium azide) and 4×10⁶ PBMCs were transferred per well in a 96-well plate. Each well was incubated with 1 μL human FcR Blocking Reagent (Miltenyi Biotec) in 50 μL FACS buffer for 15 minutes at room temperature. After the PBMCs were stained with 0.2 μL NLV-tetramer (NIH Tetramer Core Facility) (CMV pp65; amino acids ⁴⁹⁵NLVPMVATV⁵⁰³; SEQ ID NO:1) in 60 μL per well for 25 minutes at room temperature, the PBMCs were incubated with 1.4 μL anti-CD3 FITC (BD Pharmingen), 2.8 μL anti-CD4 PE/cy7 (Biolegend), and 2.8 μL anti-CD8 PerCP-cy5.5 (Biolegend) in 70 μL per well for 30 minutes at 4° C. The PBMCs were washed and the CD3+, CD4−, CD8+, NLV-tetramer+ single cells were isolated by flow cytometric sorting using a FACS Aria II (Becton Dickinson). DNA was extracted from the sorted cells using the DNEASY® Blood & Tissue kit (QIAGEN) according to the manufacturer's protocol.

Whole Genome DNA Methylation Profiling. DNA libraries were sequenced using ILLUMINA® NOVASEQ® 6000 systems. Reads were trimmed by 10 bp on 5′ and 3′ ends as well as all ILLUMINA® adapter sequences. Trimmed reads were aligned to the mm10 genome using the BSMAP v. 2.90 software (Xi & Li (2009) BMC bioinformatics 10:232).

Differential Methylation Analysis and PCA. Differential methylation analysis was carried out using R package DSS 2.34 (Wu et al. (2015) Nucleic Acids Res. 43:e141). Basic two group comparisons were run and p value threshold of 0.01 was used. DMR results with the top 150 largest absolute areaStats were used for further pathway analyses. Principal component analysis was carried out on the top 3,000 CpG sites with the highest variance. Only sites with higher than 5 reads coverage in all samples were included. The R function princomp was used to compute components.

GO Pathway Enrichment and GSEA. Gene ontology annotation was carried using Enrichr web server (Chen et al. (2013) BMC Bioinformatics 128(14); Kuleshov et al. (2016) Nucl. Acids Res. 2016:gkw377; Xie et al. (2021) Curr. Protoc. 1:e90). Input regions included the top 150 methylated and demethylated DMRs. Bar graphs were based on the top enriched GO Biological Process pathways. Pre-Ranked GSEA (Subramanian et al. (2005) Proc. Natl. Acad. Sci. USA 102:15545-15550) analysis was performed using DMR tables. Ranks for all genes were assigned by multiplying sign of log fold-change and negative log 10 of p-values. Only one DMR with the largest absolute rank were used to represent a gene. The cell cycle gene signature was selected from GOBP_CELL_CYCLE gene set in MSigDB.

Age Estimation of WGBS and Methylation Array Samples. Epigenetic ages of human samples were estimated based on DNAm PhenoAge (Levine et al. (2018) Aging (Albany NY) 10:573-591). Methylation beta values of the 513 CpG sites were calculated using methratio.py script for WGBS samples, and getBeta function of minfi R package (Aryee et al. (2014) Bioinformatics 30:1363-1369) for methylation array samples. Beta values were then multiplied with published weights and the intercept added to form age estimates. For mouse samples, epigenetic ages were estimated based on data available in the art (Lu et al. (2021) bioRxiv 2021.2001.2018.426733). Thirty-five selected CpG sites that were hypermethylated in aged individuals were used. Methylation beta values were calculated similar to human samples. Average beta values of these 35 CpGs were used to represent mouse epigenetic ages.

Publicly Available Human Cancer DNA Methylation Datasets. GRAALL Epic array data of T-ALL patients were downloaded from GEO database with accession number GSE147667 (Touzart et al. (2021) Sci. Transl. Med. 13(595):eabc4834). University of Tokyo Epic array data of T-ALL patients were downloaded from NBDC database with accession number JGAS000138 (Kimura et al. (2020) Leukemia 34:1163-1168). 450K array data of B-ALL patients were downloaded from GEO database with accession number GSE49031 (Nordlund et al. (2013) Genome Biol. 14:r105). 450K array data of AML patients were downloaded from TCGA data portal with project ID TCGA-LAML (Cancer Genome Atlas Research et al. (2013) N. Engl. J. Med. 368:2059-2074). 450K array data of melanoma patients were downloaded from GEO database with accession number GSE120878 (Conway et al. (2019) J. Invest. Dermatol. 139:1349-1361). 450K array data of neuroblastoma patients were downloaded from GEO database with GSE54719 (Gomez et al. (2015) Epigenomics 7:1137-1153). All methylation array samples were processed by Bioconductor workflow based on minfi R package. Samples were normalized with the preprocessQuantile function.

### Example 2: T Cell Epigenetic Remodeling Proceeds Beyond Organismal Lifespan Boundaries

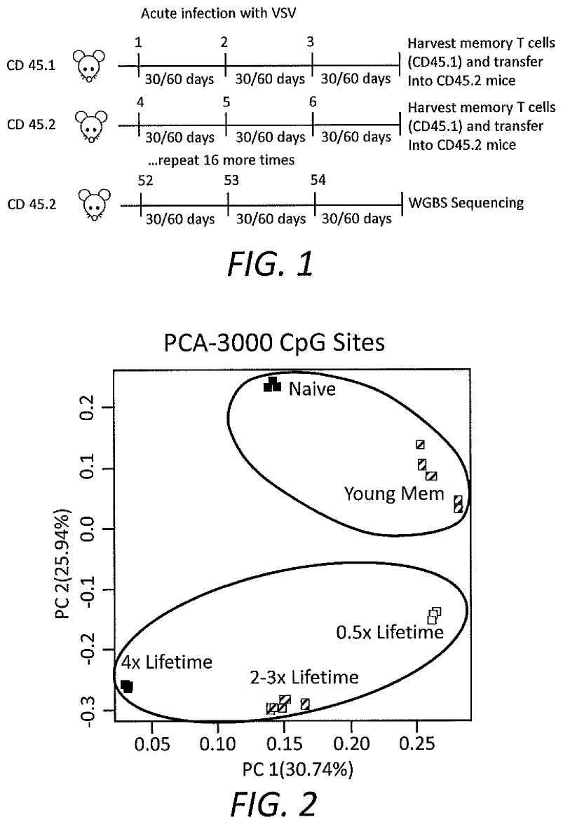

To identify a molecular signature for T cell aging that transcends organismal lifespan, an aging model was employed in which CD8 T cells far exceed the normal lifetime of the host (FIG. 1). Mice were immunized with three heterologous prime-boost-boost immunizations (VSVnj, VVn, VSVind, see Example 1) with 30-day versus 60-day intervals between infections. Congenically distinct VSV-specific CD8 T cells were then adoptively transferred to naïve mice that then underwent three additional immunizations with the heterologous virus schema. The adoptive transfer and boosting cycle were repeated up to 16 more times over 10 years to establish memory T cells ranging between 0.5 to ~4 mouse lifetimes ((herein 0.5 to 4×LT, based on an average lifespan of 2.5 years) relative to the original progenitor cells (FIG. 1). The multi-lifetime (ML) iteratively stimulated memory CD8 T cells maintain a robust ability to proliferate throughout the 51-boost regimen. Notably, the iteratively stimulated T cells preserved their proliferative capacity. Additionally, the telomere length of these ML T cells was maintained.

Using purified ML memory T cells from this model, whole genome bisulfite sequencing was performed to establish a nucleotide-resolution map of the DNA methylation changes that occur between physiologically aged and ML memory T cells. The epigenetic relationship between young memory T cells and the ML memory T cells was first assessed by performing a principal component analysis (PCA) of the top 3000 most variable CpG sites. Results from this analysis revealed a distinct segregation between young memory CD8 T cells versus the ML memory T cells, documenting CD8 T cell's ability to undergo further significant epigenetic modification throughout and far exceeding organismal lifespan. These results also indicate that analyses performed with young memory T cells fail to fully capture epigenetic programs associated with the later aging events (FIG. 2).

The genome-wide methylation distribution across T cells was determined for naïve T cells (Table 2), ‘young’ memory T cells (~3 months post infection; Table 2), advanced age memory T cells (~14 months, i.e., 0.5× lifetime; Table 3), and multiple organismal lifetime T cells including 2-3× lifetimes equivalents (Table 3) or 4× lifetime equivalents (i.e., >100 months; Table 3). Notably, a significant reduction in global DNA methylation was observed between ‘young’ memory T cells versus memory T cells analyzed at advanced age and multiple (>100 months, i.e., 4× lifetime) organismal lifetimes. Further interrogation of the DNA methylation profiles revealed epigenetic changes consistent with reinforcement of a functional CD8 T cell immune response. These changes included exaggerated DNA demethylation of effector loci and inhibitory receptors that corresponded with ATACseq-defined changes in chromatin accessibility and transcript expression.

TABLE 2

| Beta-values | |
|---|---|
| Naïve T cells | ‘Young’ Memory T cells |

| Gene | Mean | SD | Mean | SD |
|---|---|---|---|---|
| Ebf3 | 0.070 | 0.003 | 0.079 | 0.012 |
| Irx5 | 0.066 | 0.005 | 0.068 | 0.006 |
| Pou3f3 | 0.059 | 0.001 | 0.086 | 0.009 |
| Irs2 | 0.035 | 0.005 | 0.056 | 0.019 |
| Irx3 | 0.073 | 0.008 | 0.090 | 0.014 |
| Ctbp2 | 0.048 | 0.010 | 0.052 | 0.013 |
| Efna5 | 0.031 | 0.000 | 0.043 | 0.013 |
| Tle1 | 0.012 | 0.001 | 0.022 | 0.008 |
| Sall1 | 0.063 | 0.002 | 0.101 | 0.025 |
| Foxd3 | 0.080 | 0.008 | 0.108 | 0.019 |
| Zfp423 | 0.035 | 0.001 | 0.049 | 0.011 |
| Hmga2 | 0.023 | 0.005 | 0.032 | 0.007 |
| Sox1 | 0.045 | 0.004 | 0.051 | 0.009 |
| Afdn | 0.028 | 0.003 | 0.032 | 0.009 |
| Nr2f2 | 0.078 | 0.005 | 0.114 | 0.025 |
| Nkx6-1 | 0.060 | 0.003 | 0.085 | 0.026 |
| Zfp316 | 0.169 | 0.029 | 0.142 | 0.033 |
| Kctd15 | 0.041 | 0.002 | 0.046 | 0.013 |
| Sox11 | 0.069 | 0.005 | 0.151 | 0.036 |
| Sox4 | 0.012 | 0.003 | 0.031 | 0.013 |
| Spry2 | 0.027 | 0.003 | 0.031 | 0.008 |
| Cbln1 | 0.069 | 0.002 | 0.117 | 0.025 |
| Tshz3 | 0.044 | 0.003 | 0.071 | 0.023 |
| Zfp503 | 0.108 | 0.008 | 0.085 | 0.010 |
| Tfap2c | 0.080 | 0.008 | 0.090 | 0.010 |
| Inhbb | 0.063 | 0.015 | 0.080 | 0.013 |
| Irx2 | 0.079 | 0.011 | 0.134 | 0.035 |
| Gas1 | 0.033 | 0.002 | 0.091 | 0.044 |
| Fat4 | 0.053 | 0.012 | 0.064 | 0.008 |
| Nkx2-4 | 0.062 | 0.011 | 0.078 | 0.013 |
| Zcchc14 | 0.112 | 0.003 | 0.132 | 0.015 |
| Cadm1 | 0.045 | 0.005 | 0.073 | 0.018 |
| Pcdh10 | 0.068 | 0.008 | 0.119 | 0.039 |
| Onecut2 | 0.076 | 0.002 | 0.092 | 0.011 |
| Prdm6 | 0.059 | 0.009 | 0.074 | 0.017 |
| Mycn | 0.030 | 0.001 | 0.038 | 0.007 |
| Fzd8 | 0.134 | 0.019 | 0.143 | 0.013 |
| Epha7 | 0.022 | 0.003 | 0.042 | 0.018 |
| Sox9 | 0.069 | 0.002 | 0.103 | 0.021 |
| Nkx2-1 | 0.065 | 0.004 | 0.088 | 0.018 |
| Epb41l4b | 0.022 | 0.004 | 0.032 | 0.007 |
| Dio3 | 0.106 | 0.010 | 0.146 | 0.022 |
| Otx1 | 0.103 | 0.009 | 0.148 | 0.017 |
| Zbtb10 | 0.074 | 0.017 | 0.080 | 0.014 |
| Jag1 | 0.037 | 0.005 | 0.051 | 0.011 |
| Nkx6-2 | 0.081 | 0.009 | 0.090 | 0.017 |
| Tbx3 | 0.087 | 0.001 | 0.103 | 0.010 |
| Tbx18 | 0.062 | 0.001 | 0.083 | 0.016 |
| Agap1 | 0.040 | 0.005 | 0.062 | 0.015 |
| Foxc2 | 0.114 | 0.009 | 0.112 | 0.009 |
| Sox2 | 0.072 | 0.002 | 0.099 | 0.015 |
| Foxe1 | 0.080 | 0.015 | 0.097 | 0.007 |
| Fzd1 | 0.064 | 0.010 | 0.097 | 0.022 |
| Insm2 | 0.073 | 0.005 | 0.088 | 0.015 |
| Adra2c | 0.100 | 0.006 | 0.118 | 0.012 |
| Gm13375 | 0.017 | 0.002 | 0.026 | 0.007 |
| Nfib | 0.056 | 0.003 | 0.080 | 0.019 |
| Foxg1 | 0.112 | 0.010 | 0.167 | 0.027 |
| Ptpn3 | 0.022 | 0.008 | 0.019 | 0.003 |
| Mtcl1 | 0.030 | 0.006 | 0.041 | 0.009 |
| Ube2ql1 | 0.042 | 0.003 | 0.055 | 0.012 |
| Zfp536 | 0.056 | 0.004 | 0.091 | 0.018 |
| AI606473 | 0.053 | 0.002 | 0.065 | 0.010 |
| Pitx2 | 0.065 | 0.005 | 0.082 | 0.017 |
| Zfp521 | 0.016 | 0.002 | 0.022 | 0.005 |
| Grb10 | 0.065 | 0.002 | 0.085 | 0.016 |
| Lin28b | 0.033 | 0.006 | 0.056 | 0.015 |
| Gpr27 | 0.051 | 0.001 | 0.054 | 0.006 |
| Cacna2d3 | 0.055 | 0.003 | 0.075 | 0.014 |
| Zic4 | 0.096 | 0.008 | 0.106 | 0.014 |
| Fam78b | 0.031 | 0.004 | 0.051 | 0.014 |
| Begain | 0.099 | 0.019 | 0.093 | 0.016 |
| Gm9748 | 0.138 | 0.003 | 0.184 | 0.028 |
| Gm5468 | 0.044 | 0.013 | 0.054 | 0.012 |
| Prox1 | 0.071 | 0.007 | 0.111 | 0.036 |
| Schip1 | 0.036 | 0.005 | 0.043 | 0.005 |
| Bhlhe22 | 0.037 | 0.003 | 0.063 | 0.018 |
| Cdh2 | 0.062 | 0.010 | 0.107 | 0.039 |
| Fat1 | 0.047 | 0.003 | 0.088 | 0.023 |
| Twist1 | 0.047 | 0.006 | 0.060 | 0.021 |
| Ptprg | 0.065 | 0.009 | 0.104 | 0.031 |
| Sdk2 | 0.061 | 0.011 | 0.071 | 0.013 |
| Bmp4 | 0.065 | 0.005 | 0.095 | 0.023 |
| Mn1 | 0.172 | 0.038 | 0.133 | 0.043 |
| Gm30731 | 0.063 | 0.003 | 0.077 | 0.010 |
| Nova1 | 0.049 | 0.006 | 0.064 | 0.020 |
| Grid1 | 0.052 | 0.005 | 0.066 | 0.011 |
| Zfhx3 | 0.022 | 0.004 | 0.021 | 0.007 |
| Gli3 | 0.040 | 0.007 | 0.077 | 0.030 |
| Magi1 | 0.049 | 0.004 | 0.061 | 0.017 |
| Pax3 | 0.052 | 0.010 | 0.072 | 0.016 |
| Ccdc85c | 0.012 | 0.001 | 0.029 | 0.012 |
| Bnc2 | 0.037 | 0.007 | 0.064 | 0.032 |
| Mkx | 0.054 | 0.007 | 0.070 | 0.013 |
| Id4 | 0.048 | 0.002 | 0.083 | 0.027 |
| Auts2 | 0.041 | 0.009 | 0.076 | 0.025 |
| Tlx3 | 0.062 | 0.006 | 0.077 | 0.007 |
| Shisa6 | 0.068 | 0.003 | 0.127 | 0.045 |
| Ctnnd2 | 0.033 | 0.001 | 0.057 | 0.018 |
| Trank1 | 0.100 | 0.012 | 0.142 | 0.022 |
| Fam19a5 | 0.052 | 0.008 | 0.147 | 0.062 |
| Chsy3 | 0.034 | 0.001 | 0.057 | 0.016 |
| Lrp12 | 0.014 | 0.002 | 0.021 | 0.006 |
| Hoxd8 | 0.025 | 0.001 | 0.035 | 0.012 |
| Lrig1 | 0.027 | 0.005 | 0.028 | 0.008 |
| Tenm4 | 0.067 | 0.010 | 0.099 | 0.036 |
| Flt1 | 0.064 | 0.004 | 0.091 | 0.024 |
| Kcnn2 | 0.059 | 0.009 | 0.112 | 0.051 |
| Pcdh17 | 0.120 | 0.009 | 0.170 | 0.026 |
| Reln | 0.047 | 0.001 | 0.069 | 0.016 |
| Pou3f1 | 0.056 | 0.003 | 0.061 | 0.007 |
| Lrrc4 | 0.069 | 0.010 | 0.091 | 0.023 |
| Chst15 | 0.019 | 0.004 | 0.029 | 0.006 |
| Large1 | 0.029 | 0.004 | 0.054 | 0.017 |
| Fezf1 | 0.078 | 0.004 | 0.124 | 0.026 |
| Gab1 | 0.040 | 0.006 | 0.057 | 0.017 |
| Plcb4 | 0.035 | 0.013 | 0.045 | 0.026 |
| Cdh4 | 0.049 | 0.001 | 0.115 | 0.041 |
| Cilp2 | 0.281 | 0.016 | 0.304 | 0.020 |
| Prickle1 | 0.020 | 0.004 | 0.027 | 0.010 |
| Sox3 | 0.454 | 0.010 | 0.441 | 0.059 |
| Smco4 | 0.077 | 0.017 | 0.051 | 0.009 |
| Fgfr2 | 0.059 | 0.005 | 0.079 | 0.014 |
| Hoxd11 | 0.063 | 0.011 | 0.084 | 0.014 |
| Skor2 | 0.123 | 0.002 | 0.154 | 0.021 |
| Fignl2 | 0.252 | 0.009 | 0.260 | 0.049 |
| Cnr1 | 0.057 | 0.008 | 0.062 | 0.011 |
| Nbea | 0.057 | 0.003 | 0.089 | 0.028 |
| Trim71 | 0.023 | 0.004 | 0.026 | 0.005 |
| Snap91 | 0.061 | 0.004 | 0.089 | 0.019 |
| Sox14 | 0.083 | 0.005 | 0.106 | 0.022 |
| Kcnk9 | 0.109 | 0.001 | 0.150 | 0.020 |
| Mpped2 | 0.037 | 0.013 | 0.063 | 0.028 |
| Sema6a | 0.060 | 0.002 | 0.091 | 0.024 |
| Pde5a | 0.019 | 0.003 | 0.030 | 0.009 |
| Wnt5a | 0.053 | 0.005 | 0.080 | 0.018 |
| Grip1 | 0.034 | 0.006 | 0.045 | 0.016 |
| Dmrt3 | 0.039 | 0.002 | 0.048 | 0.013 |
| Adgrl3 | 0.075 | 0.034 | 0.112 | 0.038 |

SD, standard deviation

TABLE 3

| | Beta-values | | | | | |
|---|---|---|---|---|---|---|
| | 0.5X lifetime | | 2-3X lifetimes | | 4X lifetime | |
| Gene | Mean | SD | Mean | SD | Mean | SD |
| Ebf3 | 0.227 | 0.016 | 0.590 | 0.060 | 0.874 | 0.007 |
| Irx5 | 0.164 | 0.018 | 0.406 | 0.022 | 0.875 | 0.001 |
| Pou3f3 | 0.268 | 0.026 | 0.719 | 0.038 | 0.911 | 0.004 |
| Irs2 | 0.223 | 0.023 | 0.695 | 0.067 | 0.901 | 0.010 |
| Irx3 | 0.332 | 0.035 | 0.649 | 0.023 | 0.865 | 0.004 |
| Ctbp2 | 0.150 | 0.011 | 0.347 | 0.066 | 0.923 | 0.014 |
| Efna5 | 0.230 | 0.020 | 0.613 | 0.067 | 0.881 | 0.020 |
| Tle1 | 0.426 | 0.048 | 0.866 | 0.018 | 0.927 | 0.008 |
| Sall1 | 0.471 | 0.035 | 0.804 | 0.034 | 0.899 | 0.006 |
| Foxd3 | 0.297 | 0.023 | 0.736 | 0.018 | 0.875 | 0.024 |
| Zfp423 | 0.114 | 0.009 | 0.427 | 0.084 | 0.835 | 0.006 |
| Hmga2 | 0.265 | 0.024 | 0.706 | 0.047 | 0.891 | 0.026 |
| Sox1 | 0.217 | 0.031 | 0.425 | 0.052 | 0.841 | 0.043 |
| Afdn | 0.062 | 0.004 | 0.104 | 0.020 | 0.954 | 0.004 |
| Nr2f2 | 0.510 | 0.037 | 0.809 | 0.015 | 0.887 | 0.008 |
| Nkx6-1 | 0.389 | 0.015 | 0.774 | 0.014 | 0.846 | 0.008 |
| Zfp316 | 0.224 | 0.009 | 0.404 | 0.045 | 0.935 | 0.012 |
| Kctd15 | 0.111 | 0.015 | 0.316 | 0.048 | 0.902 | 0.012 |
| Sox11 | 0.596 | 0.011 | 0.860 | 0.028 | 0.919 | 0.013 |
| Sox4 | 0.159 | 0.018 | 0.476 | 0.041 | 0.845 | 0.020 |
| Spry2 | 0.214 | 0.017 | 0.706 | 0.051 | 0.875 | 0.008 |
| Cbln1 | 0.496 | 0.047 | 0.817 | 0.020 | 0.890 | 0.007 |
| Tshz3 | 0.417 | 0.030 | 0.382 | 0.049 | 0.885 | 0.007 |
| Zfp503 | 0.137 | 0.014 | 0.609 | 0.079 | 0.810 | 0.004 |
| Tfap2c | 0.160 | 0.011 | 0.401 | 0.037 | 0.877 | 0.013 |
| Inhbb | 0.236 | 0.037 | 0.449 | 0.050 | 0.888 | 0.007 |
| Irx2 | 0.560 | 0.022 | 0.843 | 0.033 | 0.914 | 0.017 |
| Gas1 | 0.603 | 0.032 | 0.887 | 0.008 | 0.915 | 0.015 |
| Fat4 | 0.172 | 0.025 | 0.622 | 0.033 | 0.892 | 0.014 |
| Nkx2-4 | 0.233 | 0.012 | 0.451 | 0.066 | 0.909 | 0.018 |
| Zcchc14 | 0.219 | 0.019 | 0.564 | 0.030 | 0.911 | 0.012 |
| Cadm1 | 0.435 | 0.023 | 0.793 | 0.033 | 0.915 | 0.014 |
| Pcdh10 | 0.489 | 0.040 | 0.802 | 0.035 | 0.897 | 0.005 |
| Onecut2 | 0.144 | 0.006 | 0.383 | 0.020 | 0.808 | 0.019 |
| Prdm6 | 0.253 | 0.029 | 0.528 | 0.048 | 0.846 | 0.005 |
| Mycn | 0.102 | 0.020 | 0.598 | 0.074 | 0.795 | 0.020 |
| Fzd8 | 0.318 | 0.018 | 0.490 | 0.056 | 0.925 | 0.014 |
| Epha7 | 0.265 | 0.023 | 0.774 | 0.042 | 0.910 | 0.011 |
| Sox9 | 0.438 | 0.016 | 0.757 | 0.038 | 0.880 | 0.023 |
| Nkx2-1 | 0.248 | 0.023 | 0.664 | 0.053 | 0.837 | 0.004 |
| Epb41l4b | 0.212 | 0.040 | 0.578 | 0.050 | 0.885 | 0.018 |
| Dio3 | 0.299 | 0.021 | 0.696 | 0.055 | 0.879 | 0.008 |
| Otx1 | 0.322 | 0.027 | 0.738 | 0.047 | 0.868 | 0.006 |
| Zbtb10 | 0.145 | 0.002 | 0.365 | 0.066 | 0.860 | 0.032 |
| Jag1 | 0.186 | 0.020 | 0.374 | 0.032 | 0.890 | 0.014 |
| Nkx6-2 | 0.185 | 0.016 | 0.404 | 0.038 | 0.939 | 0.004 |
| Tbx3 | 0.432 | 0.017 | 0.615 | 0.055 | 0.849 | 0.005 |
| Tbx18 | 0.307 | 0.037 | 0.653 | 0.042 | 0.891 | 0.009 |
| Agap1 | 0.455 | 0.053 | 0.894 | 0.021 | 0.939 | 0.011 |
| Foxc2 | 0.215 | 0.012 | 0.340 | 0.042 | 0.830 | 0.005 |
| Sox2 | 0.373 | 0.037 | 0.675 | 0.055 | 0.876 | 0.022 |
| Foxe1 | 0.244 | 0.010 | 0.508 | 0.095 | 0.812 | 0.012 |
| Fzd1 | 0.316 | 0.039 | 0.514 | 0.047 | 0.926 | 0.013 |
| Insm2 | 0.156 | 0.009 | 0.268 | 0.052 | 0.908 | 0.032 |
| Adra2c | 0.206 | 0.004 | 0.553 | 0.047 | 0.860 | 0.019 |
| Gm13375 | 0.045 | 0.006 | 0.062 | 0.009 | 0.872 | 0.019 |
| Nfib | 0.308 | 0.044 | 0.647 | 0.058 | 0.891 | 0.012 |
| Foxg1 | 0.518 | 0.018 | 0.782 | 0.059 | 0.917 | 0.003 |
| Ptpn3 | 0.222 | 0.031 | 0.424 | 0.053 | 0.906 | 0.008 |
| Mtcl1 | 0.099 | 0.008 | 0.254 | 0.034 | 0.785 | 0.041 |
| Ube2ql1 | 0.116 | 0.012 | 0.253 | 0.042 | 0.886 | 0.014 |
| Zfp536 | 0.406 | 0.028 | 0.786 | 0.044 | 0.849 | 0.025 |
| AI606473 | 0.282 | 0.033 | 0.392 | 0.040 | 0.786 | 0.020 |
| Pitx2 | 0.131 | 0.010 | 0.349 | 0.041 | 0.795 | 0.008 |
| Zfp521 | 0.102 | 0.019 | 0.147 | 0.034 | 0.832 | 0.021 |
| Grb10 | 0.290 | 0.016 | 0.779 | 0.018 | 0.916 | 0.024 |
| Lin28b | 0.212 | 0.011 | 0.774 | 0.090 | 0.926 | 0.015 |
| Gpr27 | 0.100 | 0.012 | 0.325 | 0.077 | 0.839 | 0.014 |
| Cacna2d3 | 0.263 | 0.054 | 0.643 | 0.037 | 0.896 | 0.015 |
| Zic4 | 0.287 | 0.038 | 0.670 | 0.032 | 0.850 | 0.005 |
| Fam78b | 0.245 | 0.025 | 0.835 | 0.027 | 0.905 | 0.006 |
| Begain | 0.224 | 0.002 | 0.560 | 0.085 | 0.935 | 0.016 |
| Gm9748 | 0.450 | 0.024 | 0.700 | 0.062 | 0.871 | 0.018 |
| Gm5468 | 0.364 | 0.031 | 0.805 | 0.033 | 0.889 | 0.013 |
| Prox1 | 0.445 | 0.013 | 0.749 | 0.056 | 0.859 | 0.019 |
| Schip1 | 0.101 | 0.010 | 0.386 | 0.049 | 0.886 | 0.025 |
| Bhlhe22 | 0.270 | 0.035 | 0.673 | 0.069 | 0.898 | 0.012 |
| Cdh2 | 0.440 | 0.013 | 0.834 | 0.024 | 0.905 | 0.013 |
| Fat1 | 0.428 | 0.019 | 0.708 | 0.058 | 0.870 | 0.013 |
| Twist1 | 0.336 | 0.027 | 0.722 | 0.016 | 0.857 | 0.013 |
| Ptprg | 0.450 | 0.058 | 0.800 | 0.022 | 0.873 | 0.025 |
| Sdk2 | 0.386 | 0.018 | 0.789 | 0.038 | 0.889 | 0.026 |
| Bmp4 | 0.390 | 0.015 | 0.727 | 0.024 | 0.860 | 0.019 |
| Mn1 | 0.116 | 0.014 | 0.252 | 0.006 | 0.733 | 0.015 |
| Gm30731 | 0.186 | 0.012 | 0.434 | 0.051 | 0.788 | 0.004 |
| Nova1 | 0.422 | 0.024 | 0.842 | 0.031 | 0.921 | 0.010 |
| Grid1 | 0.154 | 0.017 | 0.365 | 0.053 | 0.820 | 0.034 |
| Zfhx3 | 0.167 | 0.029 | 0.724 | 0.081 | 0.914 | 0.025 |
| Gli3 | 0.447 | 0.040 | 0.848 | 0.030 | 0.912 | 0.019 |
| Magi1 | 0.386 | 0.021 | 0.779 | 0.020 | 0.898 | 0.005 |
| Pax3 | 0.241 | 0.018 | 0.593 | 0.041 | 0.895 | 0.022 |
| Ccdc85c | 0.162 | 0.006 | 0.409 | 0.049 | 0.672 | 0.020 |
| Bnc2 | 0.427 | 0.060 | 0.822 | 0.043 | 0.921 | 0.011 |
| Mkx | 0.240 | 0.036 | 0.245 | 0.022 | 0.835 | 0.011 |
| Id4 | 0.406 | 0.027 | 0.844 | 0.035 | 0.909 | 0.008 |
| Auts2 | 0.454 | 0.048 | 0.772 | 0.016 | 0.879 | 0.007 |
| Tlx3 | 0.145 | 0.008 | 0.360 | 0.025 | 0.653 | 0.010 |
| Shisa6 | 0.620 | 0.037 | 0.878 | 0.025 | 0.929 | 0.007 |
| Ctnnd2 | 0.217 | 0.025 | 0.720 | 0.077 | 0.916 | 0.010 |
| Trank1 | 0.326 | 0.007 | 0.634 | 0.090 | 0.922 | 0.005 |
| Fam19a5 | 0.769 | 0.006 | 0.935 | 0.014 | 0.955 | 0.011 |
| Chsy3 | 0.406 | 0.030 | 0.718 | 0.067 | 0.922 | 0.012 |
| Lrp12 | 0.257 | 0.038 | 0.716 | 0.098 | 0.895 | 0.042 |
| Hoxd8 | 0.091 | 0.015 | 0.341 | 0.018 | 0.866 | 0.020 |
| Lrig1 | 0.112 | 0.001 | 0.093 | 0.018 | 0.797 | 0.020 |
| Tenm4 | 0.490 | 0.036 | 0.726 | 0.032 | 0.854 | 0.015 |
| Flt1 | 0.460 | 0.014 | 0.445 | 0.067 | 0.886 | 0.012 |
| Kcnn2 | 0.516 | 0.010 | 0.841 | 0.033 | 0.913 | 0.012 |
| Pcdh17 | 0.468 | 0.030 | 0.784 | 0.051 | 0.899 | 0.006 |
| Reln | 0.133 | 0.011 | 0.318 | 0.034 | 0.822 | 0.011 |
| Pou3f1 | 0.120 | 0.009 | 0.173 | 0.017 | 0.623 | 0.037 |
| Lrrc4 | 0.145 | 0.014 | 0.770 | 0.084 | 0.925 | 0.009 |
| Chst15 | 0.076 | 0.011 | 0.134 | 0.014 | 0.907 | 0.006 |
| Large1 | 0.303 | 0.036 | 0.789 | 0.019 | 0.870 | 0.018 |
| Fezf1 | 0.424 | 0.049 | 0.737 | 0.027 | 0.836 | 0.010 |
| Gab1 | 0.188 | 0.008 | 0.443 | 0.075 | 0.879 | 0.013 |
| Plcb4 | 0.436 | 0.069 | 0.828 | 0.039 | 0.894 | 0.014 |
| Cdh4 | 0.678 | 0.007 | 0.899 | 0.031 | 0.944 | 0.006 |
| Cilp2 | 0.404 | 0.024 | 0.597 | 0.052 | 0.918 | 0.002 |
| Prickle1 | 0.113 | 0.026 | 0.256 | 0.022 | 0.914 | 0.009 |
| Sox3 | 0.716 | 0.003 | 0.904 | 0.009 | 0.936 | 0.022 |
| Smco4 | 0.069 | 0.002 | 0.092 | 0.065 | 0.918 | 0.013 |
| Fgfr2 | 0.326 | 0.019 | 0.388 | 0.039 | 0.892 | 0.007 |
| Hoxd11 | 0.229 | 0.014 | 0.658 | 0.046 | 0.789 | 0.056 |
| Skor2 | 0.376 | 0.022 | 0.531 | 0.051 | 0.900 | 0.020 |
| Fignl2 | 0.423 | 0.011 | 0.790 | 0.044 | 0.909 | 0.020 |
| Cnr1 | 0.167 | 0.016 | 0.497 | 0.044 | 0.900 | 0.015 |
| Nbea | 0.513 | 0.056 | 0.881 | 0.015 | 0.935 | 0.029 |
| Trim71 | 0.070 | 0.005 | 0.235 | 0.019 | 0.650 | 0.009 |
| Snap91 | 0.184 | 0.004 | 0.407 | 0.026 | 0.888 | 0.009 |
| Sox14 | 0.308 | 0.023 | 0.319 | 0.016 | 0.848 | 0.036 |
| Konk9 | 0.451 | 0.024 | 0.765 | 0.043 | 0.888 | 0.019 |
| Mpped2 | 0.422 | 0.036 | 0.799 | 0.046 | 0.866 | 0.019 |
| Sema6a | 0.395 | 0.007 | 0.742 | 0.032 | 0.848 | 0.001 |
| Pde5a | 0.135 | 0.005 | 0.400 | 0.095 | 0.841 | 0.020 |
| Wnt5a | 0.354 | 0.007 | 0.702 | 0.046 | 0.873 | 0.026 |
| Grip1 | 0.182 | 0.013 | 0.400 | 0.074 | 0.867 | 0.023 |
| Dmrt3 | 0.138 | 0.040 | 0.322 | 0.039 | 0.807 | 0.020 |
| Adgrl3 | 0.533 | 0.008 | 0.831 | 0.017 | 0.880 | 0.014 |

SD, standard deviation

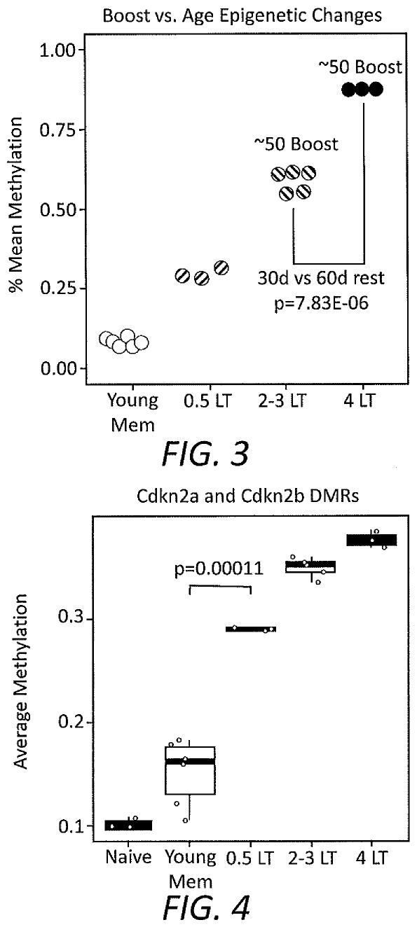

Despite a generalized genome-wide reduction in DNA methylation, discrete regions of the genome became heavily methylated in an age-associated hierarchical manner with very little change in chromatin accessibility. To further interrogate epigenetic modifications associated with cumulative age and experience despite retention of a T cell's proliferative capacity, various gene ontology and enrichment analyses (GSEA) were performed. Differentially methylated regions (DMRs) between the young and ML memory T cells revealed significant enrichment of cell cycle-related genes among the gain of methylation DMRs. Genomic annotation of the GSEA leading edges revealed that gain of methylation DMRs were highly enriched for promoter, untranslated region (UTR) and exonic regions relative to the loss of methylation DMRs. Furthermore, the gain of methylation was predominantly associated with CpG islands and shores as compared to CpG inter and shelves. Based on the genomic annotation results, attention was focused on characterization of the top methylated regions among the iteratively boosted murine memory T cells. Quantification of the overall methylation for these regions compared to the relative lifetime of the samples revealed a temporal relationship that was dependent on the age of cells (FIG. 3). Notably, T cells boosted ~50 times with 60-days rests had a highly significant greater mean methylation among these DMRs as compared to T cells boosted ~50 times with only 30 days of rest (FIG. 3). However, given that mouse memory T cells undergo a low rate of basal proliferation (often referred to as homeostatic self-renewal), this age-associated difference could be linked to cumulative proliferation. Additionally, the peak expansion of the boosted T cells was consistently much lower when the cells were only rested for 30 days, further indicating that ML memory T cells generated from 50 boosts with 30 days rest intervals underwent less proliferation relative to the cells rested for 60 days between boosts. Collectively, these results reveal that T cell age, when coupled with experience or proliferation, results in the acquisition of DNA methylation programs at cell cycle regulator loci and are correlated with a retained proliferative capacity. Moreover, by using a biological model that far exceeds organismal lifespan epigenetic programs could be defined, which were not previously discerned in CD8 T cells.

### Example 3: Supernumerary T Cell Expansion is Coupled to Epigenetic Reprogramming of Senescence-Associated Loci

Upon further examination, it was noted that cyclin-dependent kinase inhibitors (CDKs) Cdkn2a (p19^{Arf}, p16^{Ink4a}) and Cdkn2b (p15^{Ink4b}) were among the genes enriched in the methylation signature. Enrichment of DNA methylation programs at the Cdkn2a and Cdkn2b locus is notable as the encoded proteins, p19^{Arf} and p16 serve as a canonical regulators of cellular senescence, and deletion or silencing of this gene is commonly observed in various cancers, specifically malignancies of lymphoid origin. A summary of the total DMR methylation levels in the Cdkn2a/b loci shows a significant increase in average methylation levels from the young to ML memory CD8 T cells (FIG. 4). However, these DMRs are acquired in the gene body and exons, as opposed to CpG island methylation that represses expression in settings of malignancy (Gonzalez-Zulueta et al. (1995) Cancer Res. 55:4531-4535). Moreover, the notable absence of promoter methylation was accompanied by chromatin accessibility. Such exonic and gene body methylation is consistent with a transcriptional history of these canonical senescence-associated genes that likely contributes to sustained control of cell-cycle among the iteratively stimulated memory T cells while avoiding replicative senescence. Notably, gene expression from these loci was low in abundance in resting ML memory T cells. Leveraging the rich history of this senescence signaling pathway, the methylation status of other loci coding for proteins involved in regulating replicative senescence was examined. Indeed, demethylation of the oncogenes Mdm2, Rb1, and Cdk6 was inversely correlated with the gain-of-methylation observed at the Cdkn2a/2b gene cluster. Unlike the gain of methylation at the Cdkn2a/2b gene cluster, the decrease in DNA methylation of genes involved in tumor suppression was associated with an increase in chromatin accessibility. These data collectively indicate activation of these pathways to guard against malignant transformation in a setting of robust proliferation.

To further assess the T cell age epigenetic signature for genes associated with replicative senescence, the DMR gene list was cross-referenced with Oncogene Induced Senescence (OIS) specific regulatory elements (Tasdemir et al. (2016) Cancer Discov. 6:612-629). Of the top gain of methylation DMRs observed in ML T cells, over one third (49 genes) overlap with OIS activated enhancers, many of which are cell cycle regulators. Other significant genes of interest identified from the hypermethylated DMR list include those that encode additional cell cycle regulators such as Ebf3, Irx2, and Sox1. Taken together, the epigenetic modification of genes encoding cell cycle regulators (including p19^{Arf}, p16^{Ink4a}, p15^{Ink4b}) may regulate senescence of iteratively stimulated T cells and serve as a surrogate of T cell proliferation.

### Example 4: T Cell Experiential Age is Uncoupled from Organismal Chronologic Age

Given that the progressive enrichment of DNA methylation at cell-cycle regulatory genes was correlated to memory T cell age and proliferative history, it was subsequently determined whether the multi-lifetime DNA methylation signature was coupled to established epigenetic clocks. Linear regression analysis using T cells isolated from the murine ML model showed a positive correlation between the age of the cell and the average methylation of the core set of DMRs (y=0.11+0.00028x; cor=0.98). Similarly, the Horvath clock also showed a positive correlation between the age of the murine T cells and the average methylation (y=0.18+0.00015x; cor=0.93). Comparing the fit of these linear regression models showed that the mean methylation of the ML-‘experiential age’ (EA) program is better correlated with the chronologic age of the mouse T cell than the published epigenetic clock. Notably, there is little overlap between the CpG sites used for established epigenetic clocks and the EA program indicating that this T cell clock provides a novel aging metric uniquely linked to the cell's proliferative history.

It was subsequently determined whether the EA program was conserved in human settings of T cell aging. Using the St. Jude Life (SJLIFE) database of healthy individuals (Dong et al. (2022) Genome Med. 14:32), a linear regression analysis was performed between the age of the individual and the mean methylation the T cell age-associated DMRs among total PBMCs. Indeed, there was a positive correlation between age of the individual and mean methylation of the top age-associated DMRs (y=0.13+0.00025x; cor=0.54), consistent with this signal specifically coming from the T cell fraction of the total PBMC. Application of the Horvath clock yielded a stronger correlation between chronological age and estimated age, further confirming the ability of the Horvath epigenetic clock to estimate age (y=6.5+0.75x; cor=0.86). However, despite the positive correlation, the Horvath clock appeared to underestimate the host age, indicating that immune cells may age differently than the organism at-large.

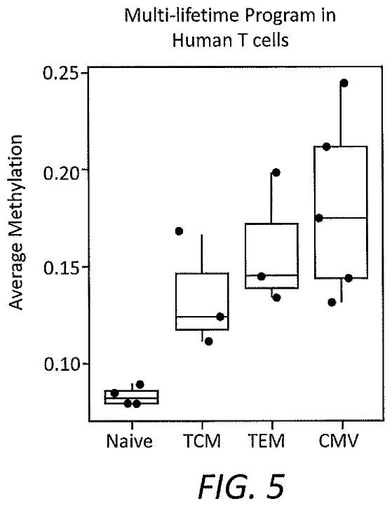

To further assess age estimates from the established epigenetic clock and the ML-EA program, analysis was focused on specific human T cell populations with known developmental states and infection history. The ML-EA program was first evaluated in well-defined human CD8 T cell subsets from healthy adults (Abdelsamed et al. (2017) J. Exp. Med. 214:1593-1606; Abdelsamed et al. (2020) Nat. Immunol. 21:578-587; Zebley et al. (2021) Cell Rep. 37:110079). Notably, central memory (Tcm), and effector memory (Tem) CD8 T cells, which would be expected to have a greater proliferative history, were significantly enriched for the ML-EA program relative to naïve CD8 T cells from the same chronologically aged donors (FIG. 5).

To further assess the relationship between the ML-EA program and T cell age, CMV-specific memory CD8 T cells were isolated from 50-60 year-old individuals. Based on their inflationary phenotype, these CMV-specific CD8 T cells reflect a human setting of repeated exposure to antigen over long periods of time, resulting in a more extreme history of iterative clonal expansion. Accordingly, the CMV-specific memory CD8 T cells had the highest levels of ML-EA-associated methylation (FIG. 5).

The Horvath epigenetic clock was subsequently applied and it was determined that indeed the CMV-specific CD8 T cells had an estimated age ranging between 20 and 50 years old consistent with the cells first encountering their cognate antigen in early life, relative to naïve CD8 T cells that had age estimates around 0 years (FIG. 5). Much like the iteratively boosted murine ML CD8 T cells, CMV-specific CD8 T cells retain control of cell-cycle and do not exhibit signs of malignant transformation. Given the prior association between CDKN2A/2B promoter silencing with malignant transformation, the methylation status of the locus in CMV-specific CD8 T cells was compared to that in a T cell leukemia. While both the leukemia and iteratively stimulated human memory T cells have methylated the gene body and exons of this locus, only the leukemia acquired methylation at the promoter region. These data indicate that EA-associated DMRs track T cell proliferation irrespective of host age and are not classical malignancy programs.

### Example 5: Human Leukemias Undergo Accelerated Epigenetic Aging

After observing that the murine-defined epigenetic hallmark of proliferative history does not include classically ascribed transformation-associated CDKN2A/2B promoter silencing, the interrogation of the T cell experiential aging signatures was expanded across multiple human settings of extreme cell division. Thus, it was determined whether the uncontrolled hyperproliferative state of various human malignancies was also coupled to epigenetic hallmarks of proliferative history. The EA signature was evaluated in publicly available DNA methylation profiles from various hematological and solid tumor patient cohorts (Touzart et al. (2021) Sci. Transl. Med. 13(595):eabc4834; Kimura et al. (2020) Leukemia 34:1163-1168; Nordlund et al. (2013) Genome Biol. 14:r105; Cancer Genome Atlas Research et al. (2013) N. Engl. J. Med. 368:2059-2074; Conway et al. (2019) J. Invest. Dermatol. 139:1349-1361). The Horvath clock was first used to establish age estimates using DNA methylation profiles from T-cell acute lymphoblastic leukemia (T-ALL), B-cell acute lymphoblastic leukemia (B-ALL), acute myeloid leukemia (AML), and melanoma patients. Notably, hematologic malignancy DNA methylation profiles yielded a significantly greater age estimation than observed with melanoma.

Given that the multi-lifetime EA signature was derived from a T cell population that underwent significant proliferation, it was determined whether T-ALL subtypes were specifically elevated in their EA estimates. Using a pediatric T-ALL cohort with patient ages ranging between 1 and 15 years old, it was first determined whether there was discordance between the patients' chronologic age and the estimated age of the patients' leukemia. Quite strikingly, several of the adolescent patients had leukemias that were estimated to be >100 years old. Furthermore, it was noted that the malignancies with an exaggerated age prediction, including HOXA and TLX3, were derived from transformation events that occurred earlier in T-cell development. Given the current clinical need for novel metrics to delineate T-ALL subtypes, the early T cell precursor subtype EA acceleration phenotype was validated using DNA methylation profiles obtained from an independent cohort at St Jude/ECOG. Again, a remarkable dichotomy in age estimates was observed among the HOXA and TLX3 subtypes versus the TAL1 subtypes, revealing leukemias derived from early developmental stages to have age estimates ranging from ~100-200 years old. Thus, well-established epigenetic clocks predict T-ALL subtypes to exhibit hallmarks of accelerated experiential aging exceeding human lifespan.

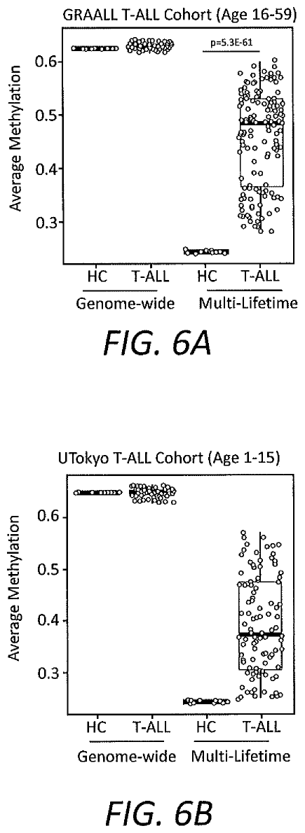

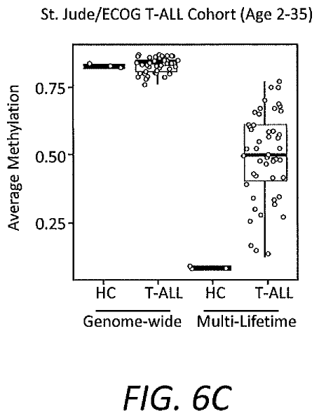

The hematological and solid tumor cancer cohorts were subsequently interrogated using the murine ML-EA methylation signature (FIG. 6). Comparison of the genome-wide average level of methylation between healthy controls (Healthy Control (HC) N=12 in FIGS. 6A and 6B; N=3 in FIG. 6C) versus the malignancy cohorts revealed that the ML-EA program was significantly enriched in the patients with hematological malignancies relative to the level of genome-wide methylation (FIG. 6). Notably, T-ALL patients had the greatest enrichment of the ML-EA program relative to the B-ALL and AML derived samples (FIG. 6). The robust enrichment of the ML-EA program among lymphocytic leukemias further documents a link between a T cells' putative proliferative history and epigenetic repression of cell-cycle regulators.

While there was indeed significant enrichment of the ML-EA program among all three independent T-ALL cohorts, each cohort possessed a striking degree of heterogeneity in the relative level of the signature (FIG. 6). To further interrogate this heterogeneity, the ML-EA program was analyzed based on the known T-ALL subtypes. Similar to the age estimate, the ML-EA program was most enriched among T-ALL derived early in T cell development, namely leukemias with HOXA and TLX3 genetic mutations. These data collectively document that the EA programs derived from the multi-lifetime murine model of memory T cell differentiation can delineate human T-ALL subtypes based on T cell maturation stage and that the accumulation of epigenetic aging metrics is uncoupled from replicative senescence.

What is claimed is:

1. A method for assessing the age and longevity of a T cell comprising determining an average methylation of a plurality of genes in the T cell, the plurality of genes selected from the group consisting of:
Ebf3, Irx5, Pou3f3, Irs2, Irx3, Ctbp2, Efna5, Tle1, Sall1, Foxd3, Zfp423, Hmga2, Sox1, Afdn, Nr2f2, Nkx6-1, Zfp316, Kctd15, Sox11, Sox4, Spry2, Cbln1, Tshz3, Zfp503, Tfap2c, Inhbb, Irx2, Gas1, Fat4, Nkx2-4, Zcchc14, Cadm1, Pcdh10, Onecut2, Prdm6, Mycn, Fzd8, Epha7, Sox9, Nkx2-1, Epb4114b, Dio3, Otx1, Zbtb10, Jag1, Nkx6-2, Tbx3, Tbx18, Agap1, Foxc2, Sox2, Foxe1, Fzd1, Insm2, Adra2c, Gm13375, Nfib, Foxg1, Ptpn3, Mtcl1, Ube2ql1, Zfp536, AI606473, Pitx2, Zfp521, Grb10, Lin28b, Gpr27, Cacna2d3, Zic4, Fam78b, Begain, Gm9748, Gm5468, Prox1, Schip1, Bhlhe22, Cdh2, Fat1, Twist1, Ptprg, Sdk2, Bmp4, Mn1, Gm30731, Nova1, Grid1, Zfhx3, Gli3, Magi1, Pax3, Ccdc85c, Bnc2, Mkx, Id4, Auts2, Tlx3, Shisa6, Ctnnd2, Trank1, Fam19a5, Chsy3, Lrp12, Hoxd8, Lrig1, Tenm4, Flt1, Kcnn2, Pcdh17, Reln, Pou3f1, Lrrc4, Chst15, Large1, Fezf1, Gab1, Plcb4, Cdh4, Cilp2, Prickle1, Sox3, Smco4, Fgfr2, Hoxd11, Skor2, Fignl2, Cnr1, Nbea, Trim71, Snap91, Sox14, Kcnk9, Mpped2, Sema6a, Pde5a, Wnt5a, Grip1, Dmrt3, and Adgrl3,
wherein an average hypermethylation of the plurality of genes in the T cell as compared to a control naïve T cell is indicative of the age and longevity of the T cell.

2. The method of claim 1, wherein the T cell is a memory T cell.

3. The method of claim 1, wherein the methylation of the plurality of genes is determined in the gene body of the plurality of genes.

4. The method of claim 1, wherein the methylation of the plurality of genes is determined in CpG islands and shores of the plurality of genes.

5. A method for selecting a T cell for a T cell therapy comprising determining an average methylation of a plurality of genes in the T cell, the plurality of genes selected from the group consisting of:
Ebf3, Irx5, Pou3f3, Irs2, Irx3, Ctbp2, Efna5, Tle1, Sall1, Foxd3, Zfp423, Hmga2, Sox1, Afdn, Nr2f2, Nkx6-1, Zfp316, Kctd15, Sox11, Sox4, Spry2, Cbln1, Tshz3, Zfp503, Tfap2c, Inhbb, Irx2, Gas1, Fat4, Nkx2-4, Zcchc14, Cadm1, Pcdh10, Onecut2, Prdm6, Mycn, Fzd8, Epha7, Sox9, Nkx2-1, Epb41l4b, Dio3, Otx1, Zbtb10, Jag1, Nkx6-2, Tbx3, Tbx18, Agap1, Foxc2, Sox2, Foxe1, Fzd1, Insm2, Adra2c, Gm13375, Nfib, Foxg1, Ptpn3, Mtcl1, Ube2ql1, Zfp536, AI606473, Pitx2, Zfp521, Grb10, Lin28b, Gpr27, Cacna2d3, Zic4, Fam78b, Begain, Gm9748, Gm5468, Prox1, Schip1, Bhlhe22, Cdh2, Fat1, Twist1, Ptprg, Sdk2, Bmp4, Mn1, Gm30731, Nova1, Grid1, Zfhx3, Gli3, Magi1, Pax3, Ccdc85c, Bnc2, Mkx, Id4, Auts2, Tlx3, Shisa6, Ctnnd2, Trank1, Fam19a5, Chsy3, Lrp12, Hoxd8, Lrig1, Tenm4, Flt1, Kcnn2, Pcdh17, Reln, Pou3f1, Lrrc4, Chst15, Large1, Fezf1, Gab1, Plcb4, Cdh4, Cilp2, Prickle1, Sox3, Smco4, Fgfr2, Hoxd11, Skor2, Fignl2, Cnr1, Nbea, Trim71, Snap91, Sox14, Kcnk9, Mpped2, Sema6a, Pde5a, Wnt5a, Grip1, Dmrt3, and Adgrl3,
wherein an average hypermethylation of the plurality of genes in the T cell as compared to a control naïve T cell is indicative of the use of the T cell in a T cell therapy.

6. The method of claim 5, wherein the T cell is a memory T cell.

7. The method of claim 5, wherein the methylation of the plurality of genes is determined in the gene body of the plurality of genes.

8. The method of claim 5, wherein the methylation of the plurality of genes is determined in CpG islands and shores of the plurality of genes.

9. The method of claim 5, wherein the T cell has at least one edited gene.

10. A method for determining a T-cell acute lymphoblastic leukemia (T-ALL) subtype comprising determining an average methylation of a plurality of genes of the T-ALL cells, the plurality of genes selected from the group consisting of:
Ebf3, Irx5, Pou3f3, Irs2, Irx3, Ctbp2, Efna5, Tle1, Sall1, Foxd3, Zfp423, Hmga2, Sox1, Afdn, Nr2f2, Nkx6-1, Zfp316, Kctd15, Sox11, Sox4, Spry2, Cbln1, Tshz3, Zfp503, Tfap2c, Inhbb, Irx2, Gas1, Fat4, Nkx2-4, Zcchc14, Cadm1, Pcdh10, Onecut2, Prdm6, Mycn, Fzd8, Epha7, Sox9, Nkx2-1, Epb41l4b, Dio3, Otx1, Zbtb10, Jag1, Nkx6-2, Tbx3, Tbx18, Agap1, Foxc2, Sox2, Foxe1, Fzd1, Insm2, Adra2c, Gm13375, Nfib, Foxg1, Ptpn3, Mtcl1, Ube2ql1, Zfp536, AI606473, Pitx2, Zfp521, Grb10, Lin28b, Gpr27, Cacna2d3, Zic4, Fam78b, Begain, Gm9748, Gm5468, Prox1, Schip1, Bhlhe22, Cdh2, Fat1, Twist1, Ptprg, Sdk2, Bmp4, Mn1, Gm30731, Nova1, Grid1, Zfhx3, Gli3, Magi1, Pax3, Ccdc85c, Bnc2, Mkx, Id4, Auts2, Tlx3, Shisa6, Ctnnd2, Trank1, Fam19a5, Chsy3, Lrp12, Hoxd8, Lrig1, Tenm4, Flt1, Kcnn2, Pcdh17, Reln, Pou3f1, Lrrc4, Chst15, Large1, Fezf1, Gab1, Plcb4, Cdh4, Cilp2, Prickle1, Sox3, Smco4, Fgfr2, Hoxd11, Skor2, Fignl2, Cnr1, Nbea, Trim71, Snap91, Sox14, Kcnk9, Mpped2, Sema6a, Pde5a, Wnt5a, Grip1, Dmrt3, and Adgrl3,
wherein the average methylation of the plurality of genes in the T cell as compared to a control naïve T cell is indicative of the T-ALL subtype.

11. The method of claim 10, wherein the T cell is a memory T cell.

12. The method of claim 10, wherein the methylation of the plurality of genes is determined in the gene body of the plurality of genes.

13. The method of claim 12, further comprising determining a methylation status of a Cdkn2a or Cdkn2b promoter.

14. The method of claim 10, wherein the methylation of the plurality of genes is determined in CpG islands and shores of the plurality of genes.

15. The method of claim 10, wherein the T-ALL subtype is a HOXA or TLX3 subtype.


SEQUENCE LISTING (1 sequence)

SEQ ID NO: 1 - protein, 9 aa, Synthetic construct
NLVPMVATV